(19) United States
(12) Patent Application Publication
(10) Pub. No.: US 2021/0015974 A1
(43) Pub. Date: Jan. 21, 2021

(54) BIODEGRADABLE POLYMERIC FILM INCLUDING EXTRACELLULAR MATRIX AND USE THEREOF

(71) Applicant: KOREA INSTITUTE OF SCIENCE AND TECHNOLOGY, Seoul (KR)

(72) Inventors: Kwi Deok PARK, Seoul (KR); Jong Ho LEE, Seoul (KR); Eui Sun SONG, Seoul (KR); Yoon Ki JOUNG, Seoul (KR)

(21) Appl. No.: 16/919,111

(22) Filed: Jul. 2, 2020

(30) Foreign Application Priority Data
Jul. 19, 2019 (KR) 10-2019-0087755

(51) Int. Cl. A61L 27/36; A61L 27/58; A61L 27/18; A61L 27/54

(52) U.S. Cl. CPC A61L 27/3687; A61L 27/58; A61L 27/18; A61L 2300/412; A61L 27/3633; A61L 2430/16; A61L 27/54

(57) ABSTRACT

Provided are a biodegradable polymeric film including an extracellular matrix, and use thereof, and particularly, a method of producing a poly(lactide-co-ε-caprolactone) film including an extracellular matrix, a film produced by the method, and an ophthalmic material including the film.

### CROSS-REFERENCE TO RELATED APPLICATION

This application claims the benefit of Korean Patent Application No. 10-2019-0087755, filed on Jul. 19, 2019, in the Korean Intellectual Property Office, the disclosure of which is incorporated herein in its entirety by reference.

### BACKGROUND

### 1. Field

The present disclosure relates to a biodegradable polymeric film including an extracellular matrix, and use thereof. This application claims the benefit of Korean Patent Application No. 10-2019-0087755, filed on Jul. 19, 2019, in the Korean Intellectual Property Office, the disclosure of which is incorporated herein in its entirety by reference.

### 2. Description of Related Art

Biocompatible polymeric materials are widely applied to clinical trials as a means to diagnose, treat, and prevent diseases, and particularly, used as a basic material for artificial organs and artificial tissues that may be used to replace damaged or missing human tissues and organs. The artificial organs include artificial hearts, kidneys, cardiopulmonary machines, blood vessels, etc., and the artificial tissues include artificial cartilage, artificial bones, artificial skin, artificial tendons, etc. In addition, biocompatible polymeric materials are also used in dental materials, sutures, polymeric drugs, etc.

Such biocompatibility may have different meanings in two aspects. Biocompatibility in a broad sense means having a desired function and safety for a living body, and biocompatibility in a narrow sense means having biological safety for a living body, that is, having no toxicity and being sterilizable. Thus, a biocompatible polymer refers to a polymer that exhibits a desired function in the body and is itself non-toxic as well as sterilizable. However, if a cell surface receptor does not recognize a polymer surface during cell attachment, a decrease in an effective value of the biocompatible polymer material is unavoidable. In recent years, studies have been conducted to increase cell affinity by treating the polymer surface with natural polymers such as peptides, fibronectin, vitronectin, and laminin, which are related to cell attachment. These methods may be effective in attachment and amplification of cells to a certain degree, but cannot be considered to be surface microenvironments close to being biomimetic, which is a step for the cells to actually recognize as their natural environment. Therefore, the development of biocompatible structures and supports that are most close to the actual cell environment are needed (Korean Patent Publication No. 10-2015-0128481).

Meanwhile, an extracellular matrix (ECM) obtained by culturing body tissues or cells is one of biomaterials that best realizes the cellular microenvironment. In existing technologies, examples of tissue ECM obtained by acellularizing cells from live allogenic or xenogenic tissues are small intestinal submucosa (SIS), urinary bladders (UBS), human amniotic membrane (HAM), or Achilles tendon as its main source, and may be applied in various types of 3-dimensional support based on its excellent physical properties. Meanwhile, an ECM-based structure derived from cells is free from an immune reaction through autologous cell culture, and an ECM structure synthesized by the cells themselves provides a physical topographical cue related to cell attachment, which is effective in migration and amplification of cells. Also, various macromolecular components such as collagen, fibronectin, or laminin in the ECM provide a biochemical microenvironment and thus may act favorably for differentiation into particular cells. However, due to the weak physical properties of being easily torn and broken despite excellent biological effects, there have been considerable limitations in application to the body and in production and application of a 3-dimensional structure. Also, in terms of existing technologies, after cell culture, it is not possible to detach the ECM from a culture plate through an acellularizing process without collapse of the shape of the ECM. The detachment of the ECM has only reached the level of physically scraping out the ECM by using a cell scraper. This ultimately destroys a self-assembled ECM structure and thus has a technical weakness in that the morphological advantages of the ECM may not be completely realized.

In view of this technical background, the present inventors have made intensive efforts to develop a polymer-derived material having excellent biocompatibility and cell amplification ability, and as a result, have prepared a material in a form in which extracellular matrix components are linked to a poly(lactide-co-ε-caprolactone) polymer via physical crosslinking, thereby completing the present disclosure.

### SUMMARY

An aspect provides a method of producing a poly(lactide-co-ε-caprolactone) film including an extracellular matrix.

Another aspect provides a poly(lactide-co-ε-caprolactone) film including an extracellular matrix, which is produced by the above method.

Still another aspect provides a method of regenerating a biological tissue, the method including administering or implanting, into an individual, the poly(lactide-co-ε-caprolactone) film including an extracellular matrix, which is produced by the above method.

Still another aspect provides an ophthalmic material including the poly(lactide-co-ε-caprolactone) film including an extracellular matrix.

Other objects of the present disclosure and the including of this extracellular matrix will be further clarified by the following detailed description together with the appended claims. Since contents that are not described in the present disclosure may be sufficiently recognized and inferred by those skilled in the art or similar art, a description thereof will be omitted.

Additional aspects will be set forth in part in the description which follows and, in part, will be apparent from the description, or may be learned by practice of the presented embodiments of the disclosure.

An aspect provides a method of producing a poly(lactide-co-ε-caprolactone) film including an extracellular matrix (ECM), the method including producing a two-phase mixture by adding a poly(lactide-co-ε-caprolactone)(PLCL) polymer solution to a solid-phase substrate to which ECM is attached; and forming a PLCL-ECM film on the surface of the solid-phase substrate by evaporating the solvent from the produced two-phase mixture,

wherein the PLCL-ECM film is formed by physical crosslinking between the PLCL and the ECM.

Each step of the method of producing the PLCL film including ECM will be described in detail below.

The method may include producing a two-phase mixture by adding a PLCL polymer solution to a solid-phase substrate to which ECM is attached, thereby forming two phases while PLCL comes in direct contact with ECM at the interface.

As used herein, the term “ECM” refers to a tissue that is responsible for structural support of animals (not humans?), etc., and belongs to a connective tissue. ECM consists of an interstitial matrix that fills the space between many cells, and a basement membrane. ECM is physically and firmly attached to the polymer surface and serves to mimic in vivo microenvironment. For example, ECM may influence attachment, survival, and proliferation of cells that exist around the area where the film is attached.

In a specific embodiment, the ECM may be obtained through decellularization of a biological tissue or a cultured cell layer. Further, the ECM may be obtained through decellularization of a cultured fibroblast cell layer, for example, through decellularization of a human lung fibroblast cell layer. The term “decellularization” refers to a process of removing cellular components from a tissue or a cell layer, which remains an ECM. The decellularization may be performed by contacting an alkali, DNase, or RNase with the tissue or the cell layer. In addition, the decellularization may be performed by applying a common method known in the art.

Thus, the PLCL polymer may refer to a copolymer having hydrophobicity, which is composed of a lactide unit and a caprolactone unit. In a specific embodiment, the PLCL polymer may have a molecular weight of 100 kDa to 200 kDa. The molecular weight of the PLCL polymer may be, for example, 100 kDa to 190 kDa, 100 kDa to 170 kDa, 100 kDa to 150 kDa, 100 kDa to 130 kDa, 100 kDa to 110 kDa, 110 kDa to 200 kDa, 110 kDa to 190 kDa, 110 kDa to 170 kDa, 110 kDa to 150 kDa, 110 kDa to 130 kDa, 120 kDa to 200 kDa, 120 kDa to 190 kDa, 120 kDa to 170 kDa, 120 kDa to 150 kDa, 120 kDa to 130 kDa, 130 kDa to 200 kDa, 130 kDa to 190 kDa, 130 kDa to 170 kDa, 130 kDa to 150 kDa, 140 kDa to 200 kDa, 140 kDa to 190 kDa, 140 kDa to 170 kDa, or 140 kDa to 150 kDa.

In the PLCL solution, a solvent may be any solvent in which PLCL may be dissolved. In a specific embodiment, the solvent may be an aqueous buffer solution such as water, a phosphate buffered solution (PBS), distilled water, and saline, and an organic solvent such as chloroform, tetrahydrofuran, hexafluoroisopropanol, dimethylformamide, acetone, and dimethyl sulfoxide (DMSO). The PLCL solution may have a concentration of 0.5% (w/v) to 5% (w/v). The concentration of the PLCL solution may be, for example, 0.5% (w/v) to 4.5% (w/v), 0.5% (w/v) to 4.0% (w/v), 0.5% (w/v) to 3.5% (w/v), 0.5% (w/v) to 3.0% (w/v), 0.5% (w/v) to 2.5% (w/v), 0.5% (w/v) to 2.0% (w/v), 0.5% (w/v) to 1.5% (w/v), 1% (w/v) to 4.5% (w/v), 1% (w/v) to 4.0% (w/v), 1% (w/v) to 3.5% (w/v), 1% (w/v) to 3.0% (w/v), 1% (w/v) to 2.5% (w/v), 1% (w/v) to 2.0% (w/v), 1% (w/v) to 1.5% (w/v), 2% (w/v) to 5.0% (w/v), 2% (w/v) to 4.5% (w/v), 2% (w/v) to 4.0% (w/v), 2% (w/v) to 3.5% (w/v), 2% (w/v) to 3.0% (w/v), 2% (w/v) to 2.5% (w/v), 3% (w/v) to 5.0% (w/v), 3% (w/v) to 4.5% (w/v), 3% (w/v) to 4.0% (w/v), or 3% (w/v) to 3.5% (w/v).

The two-phase mixture is formed on the solid-phase substrate, such as cover-slip glass, etc., and the added PLCL solution is applied onto the ECM to form the two phases. In a specific embodiment, the two phases may form a separate layer while the ECM layer comes in close contact with the PLCL polymer solution at the interface therebetween.

Thereafter, the method may include forming a PLCL-ECM film on the surface of the solid-phase substrate by evaporating the solvent from the two-phase mixture produced in the above process.

In a specific embodiment, the evaporation of the solvent may be carried out at 5° C. to 60° C. The temperature may be, for example, 5° C. to 55° C., 5° C. to 45° C., 5° C. to 35° C., 5° C. to 25° C., 5° C. to 15° C., 15° C. to 55° C., 15° C. to 45° C., 15° C. to 35° C., or 15° C. to 25° C., but it may be appropriately changed depending on the purpose of use and the type of the solvent.

In a specific embodiment, the evaporation of the solvent may be carried out for 12 hr to 72 hr. The condition may be 12 hr to 60 hr, 12 hr to 48 hr, 12 hr to 36 hr, 12 hr to 24 hr, 24 hr to 72 hr, 24 hr to 60 hr, 24 hr to 48 hr, or 24 hr to 36 hr, but it may be appropriately changed, as long as sufficient physical crosslinking may occur between PLCL and ECM under the condition.

In a specific embodiment, when the solvent is chloroform, the evaporating may be carried out at room temperature for 12 hr to 36 hr.

In the above process, the solvent of the PLCL solution is evaporated, and as the contact area between the PLCL polymer and the ECM is increased, physical crosslinking between the PLCL polymer and the ECM may be enhanced or reinforced.

Thereafter, the method may further include separating the PLCL-ECM film from the surface of the solid-phase substrate. The process may be to physically separate the PLCL-ECM film from the surface of the solid-phase substrate, for example, by gripping a portion of the cross-linked PLCL-ECM film, and then by pulling in a direction away from the surface of the solid-phase substrate. As needed, the method may further include molding the separated PLCL-ECM film, which may be carried out by a common method known in the art.

Further, the method may further include adding, to the PLCL film including ECM, cells to be implanted or administered. For example, cells to be implanted or administered may be dispensed on the ECM side of the film produced by the method, and thus stably attached thereto, which may be carried out by a common method known in the art.

Another aspect provides a PLCL film including ECM, which is produced by the above method.

Still another aspect provides a method of regenerating a biological tissue, the method including administering or implanting, into an individual, the PLCL film including ECM, which is produced by the above method.

Of the terms or elements mentioned in the PLCL film including ECM, those the same as mentioned in the description of the production method are as described above.

As used herein, the term “individual” refers to a subject in need of treatment of a disease, or regeneration of a biological tissue, and more specifically, mammals such as human or non-human primate, mouse, dog, cat, horse, cattle, etc.

According to one exemplary embodiment, the film produced according to the above method may be a PLCL-ECM film formed through a physical crosslinking reaction between PLCL and ECM, and the PLCL-ECM film may maintain transparency originating from the existing material, while stably attaching ECM to the surface of the polymer. Therefore, the PLCL-ECM film has functions of providing conditions that mimic the in vivo microenvironment for cells existing around the film and exerting excellent biological functions.

In a specific embodiment, the film may have a sheet or film shape in a transparent state. The film may have a thickness of 5 μm to 25 μm. The thickness of the film may be, for example, 5 μm to 20 μm, 5 μm to 15 μm, 5 μm to 10 μm, 10 μm to 25 μm, 10 μm to 20 μm, 10 μm to 15 μm, or 15 μm to 25 μm, or 15 μm to 20 μm.

In a specific embodiment, the administering or implanting into an individual may be appropriately changed according to a target tissue, and a technology known in the art may be applied without limitation. For example, when the PLCL film including ECM is applied to the eye, the film may be implanted in the form of a contact lens, an artificial lens, and an artificial cornea known in the art.

In a specific embodiment, ECM may be attached onto the surface of the film. As described above, ECM attached onto the surface of the film may influence survival and proliferation of cells that exist around the area where the film is attached, and thus it may be used as an implant material for in vitro cell culture or biological tissue regeneration. Further, the film may be used as a patch for wound healing, as a cardiac patch, or as a patch for the treatment of diabetic foot ulcers, etc., and in particular, also as an ophthalmic material.

Still another aspect provides an ophthalmic material including the PLCL film including ECM.

Of the terms or elements mentioned in the ophthalmic material, those the same as mentioned in the description of the production method and the film are as described above.

According to an exemplary embodiment, since the PLCL film including ECM may significantly enhance attachment, survival, and proliferation of human corneal endothelial cells, the film may be used as an ophthalmic material for tissue regeneration.

The ophthalmic material may be a contact lens. The ophthalmic material may be used as a component of an artificial lens, an artificial cornea, an artificial eye, etc., and may be formulated in the form of an eye patch. As needed, the ophthalmic material may further include a commonly used ophthalmic drug, which may be carried out through a common method known in the art.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 shows a schematic illustration of a process of producing a poly(lactide-co-ε-caprolactone) film (PLCL-ECM film) including an extracellular matrix according to one exemplary embodiment;

FIGS. 2A and 2B show physical properties (e.g., transparency) of the PLCL-ECM film according to one exemplary embodiment, wherein FIG. 2A shows results of visual observation of the PLCL-ECM film, and FIG. 2B shows results of examining the thickness of a cross-section of the PLCL-ECM film with a scanning electron microscope;

FIG. 3 shows results of using a confocal laser microscope to observe the PLCL-ECM film according to one exemplary embodiment immunostained with a fibronectin (FN) antibody, to identify the ECM existing on the PLCL-ECM film;

FIG. 4 shows results of a live & dead assay to examine the effect of the PLCL-ECM film according to one exemplary embodiment on viability of WI-38 cells;

FIG. 5 shows results of examining cell attachment through intracellular F-actin and vinculin immunostaining afte seeding human corneal endothelial cells (hCECs) on the PLCL-ECM film according to one exemplary embodiment;

FIG. 6 shows results of examining the effects of the PLCL-ECM film according to one exemplary embodiment on proliferation of hCEC cells through a CCK-8 assay, as compared with a fibronectin-coated group (PLCL-FN); and

FIG. 7 shows results of comparing the cell proliferation effects between the PLCL-ECM film according to one exemplary embodiment and a PVA-ECM film, wherein the proliferation of NIH3T3 cells was compared through a CCK-8 assay.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Hereinafter, the present disclosure will be described in more detail with reference to exemplary embodiments. However, these exemplary embodiments are only for illustrating the present disclosure, and the scope of the present disclosure is not limited to these exemplary embodiments.

### Example 1. Production of Poly(Lactide-Co-ε-Caprolactone) Film Including Extracellular Matrix

1-1. Preparation of Human Lung Fibroblast-Derived Matrix

A human lung fibroblast WI-38 cell line (ATCC CCL-75) was seeded at a density of 2×10⁴ cells/cm² on a cover slip glass (18 mm). Dulbecco's modified Eagle's medium (DMEM) supplemented with 10% (v/v) fetal bovine serum (FBS), 100 U/ml penicillin, and 100 μl/ml streptomycin was added to the cover slip glass on which the human lung fibroblast WI-38 cell line was seeded, and cultured for about 7 days under general culture conditions (5% CO₂, 37° C.) while replacing the medium every 2 or 3 day. Thereafter, the cultured cells were washed with phosphate-buffered saline (PBS). Subsequently, 0.25% (v/v) Triton-X 100 and 50 mM NH₄OH (Sigma) were added to the washed cells, and then 50 U/ml of DNase I (Invitrogen) and 2.5 μl/ml of RNase A (Invitrogen) were added, and decellularization was carried out by incubation at 37° C. for 2 hr. Thereafter, the decellularized extracellular matrix (ECM) was washed with PBS to obtain a human lung fibroblast-derived matrix (hFDM). The obtained human lung fibroblast-derived matrix was used immediately or stored at about 4° C. in the presence of PBS until use.

1-2. Preparation of Poly(Lactide-Co-ε-Caprolactone) Solution

A poly(lactide-co-ε-caprolactone) (PLCL, molecular weight: about 138 kDa, Resormer LC 703, Evonik) copolymer and chloroform (molecular weight: about 119.38, Sigma) were stirred using a magnetic stirrer at 500 rpm for 4 hr, and homogeneously dissolved, and as a result, 2.5% (w/v) of a poly(lactide-co-ε-caprolactone) polymer solution (hereinafter, referred to as a PLCL solution) was prepared using chloroform as a solvent.

1-3. Production of Poly(Lactide-Co-ε-Caprolactone) Film Including Extracellular Matrix

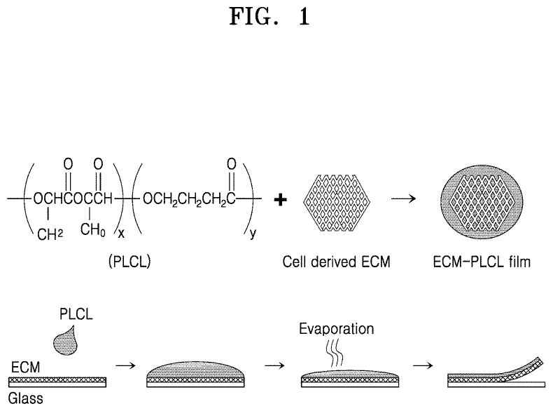

100 μl of 2.5% (w/v) PLCL solution was placed onto the human lung fibroblast-derived matrix prepared in Example 1-1, and exposed at room temperature for 24 hr to fully evaporate the chloroform solvent. During the process, a physical crosslinking reaction between PLCL and human lung fibroblast-derived matrix was induced, and finally, a poly(lactide-co-ε-caprolactone) film including ECM (hereinafter, referred to as a PLCL-ECM film) was developed, as shown in FIG. 1. Thereafter, distilled water was added to the PLCL-ECM film, and left for 5 min. Then, the physically crosslinked PLCL-ECM film was carefully detached from the cover slip glass using a forceps, and transferred to a new plate to reserve a PLCL-ECM film according to one exemplary embodiment.

### Example 2. Examination of Physical and Surface Properties of PLCL-ECM Film

In this exemplary embodiment, physical properties of the PLCL-ECM film produced in Example 1 were examined, and it was also examined whether the human lung fibroblast-derived matrix which is an ECM component was actually attached on the surface of the film. In detail, appearance of the PLCL-ECM film was visually observed. Thereafter, the PLCL-ECM film was cut with a surgical knife, and the cross-section was observed with a scanning electron microscope. Further, the PLCL-ECM film was immunostained using an anti-fibronectin antibody (catalog no. SC-8422, Santa Cruz Biotechnology) as a primary antibody, and Alexa Fluor 488-conjugated anti-mouse IgG antibody as a secondary antibody, and the PLCL-ECM film specifically immunostained with fibronectin was observed with a confocal laser microscope (Zeiss, LSM700).

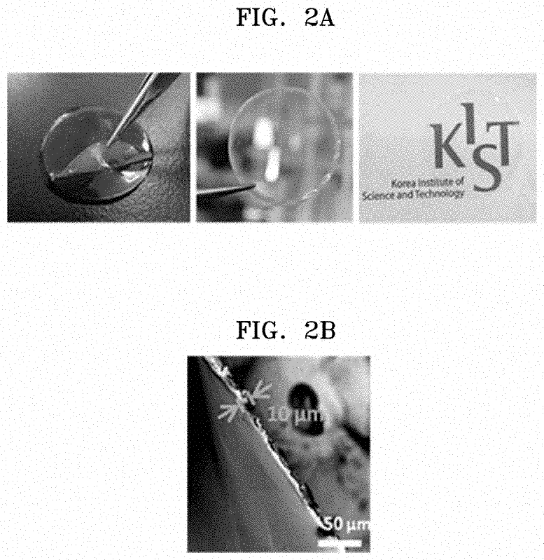

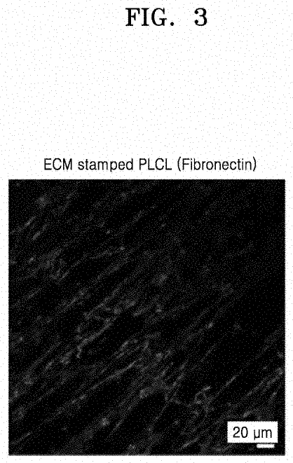

As a result, as shown in FIGS. 2A and 2B, the PLCL-ECM film was found to be a transparent film having a thickness of about 10 μm. Further, as shown in FIG. 3, it was found that a large amount of the human lung fibroblast-derived matrix was present on the surface of the film separated from the cover slip glass. In other words, these experimental results indicate that the human lung fibroblast-derived matrix present on the surface of the film was attached securely on the PLCL film while well maintaining its original fiber structure, and the polymer film having the above-described physical properties, i.e., thin and transparent properties shows its applicability as an ophthalmic material.

### Example 3. Examination of Effect of PLCL-ECM Film on In-Vivo (In Vitro?) Cells

3-1. Evaluation of Biocompatibility

To examine biocompatibility of the PLCL-ECM film, WI-38 cells were dispensed at a density of 1×10⁴ cells/ml on the PLCL-ECM film, and cultured for 24 hr to evaluate viability of the cells by a live & dead assay. In detail, the PLCL-ECM film including WI-38 cells was washed with a Dulbecco's phosphate-buffered saline (DPBS) solution (Sigma-Aldrich), and then co-treated with calcein AM (green) and ethidium bromide (red), and then incubated for 30 min to evaluate cell viability. At this time, live cells were stained green and dead cells were stained red.

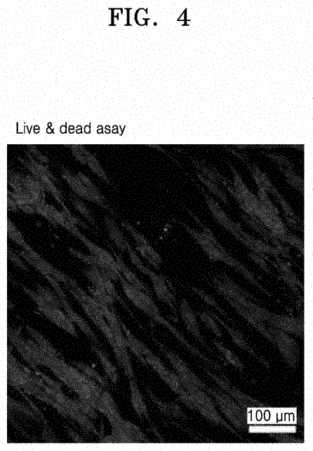

As a result, as shown in FIG. 4, most of WI-38 cells present on the PLCL-ECM film were stained green, and cells stained red were rarely observed. These experimental results indicate that the PLCL-ECM film according to one exemplary embodiment has excellent biocompatibility.

3-2. Evaluation of Cell Adhesion Ability

To examine cell adhesion ability of the PLCL-ECM film, human corneal endothelial cells (hCECs) were dispensed at a density of 1×10⁴ cells/ml on the PLCL-ECM film, and cultured for 24 hr. The PLCL-ECM film including hCECs was washed with a DPBS solution, and then subjected to immunofluorescence staining to examine expression of cell adhesion proteins. In the immunofluorescence staining, the cell nuclei were stained with DAPI (blue), and F-actin and vinculin which are cell adhesion markers were stained with Alexa Fluor® 594 (red) and Alexa Fluor® 488, respectively.

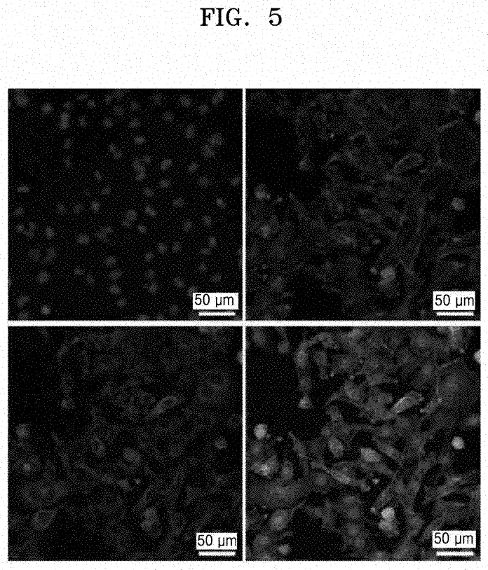

As a result, as shown in FIG. 5, both F-actin and vinculin were observed in hCECs cultured on the PLCL-ECM film, consistent with the hCECs distribution on the film as identified via DAPI staining. These experimental results indicate that the PLCL-ECM film according to one exemplary embodiment is able to stably attach human corneal endothelial cells.

3-3. Evaluation of Cell Proliferation Ability

To examine cell proliferation ability on the PLCL-ECM film, cultured hCECs were subjected to a CCK-8 assay. In detail, the PLCL-ECM film including hCECs at a density of 1×10⁴ cells/ml was washed with a DPBS solution, and then 500 μl of medium was added thereto, and 50 μl of water-soluble tetrazolium salt-8 (WST-8) was added to the cell-dispensed solution. Thereafter, cells were incubated for 2 hr in the dark, and then absorbance at 450 nm was measured by an ELISA reader. The measurement was expressed as a cell proliferation rate (%), based on a level of the cells present on the fibronectin (FN)-coated PLCL film (PLCL-FN) on day 0, and the above measurement was performed on day 2 and 5 after culture. As a control, an FN-coated PLCL film (PLCL-FN) was used.

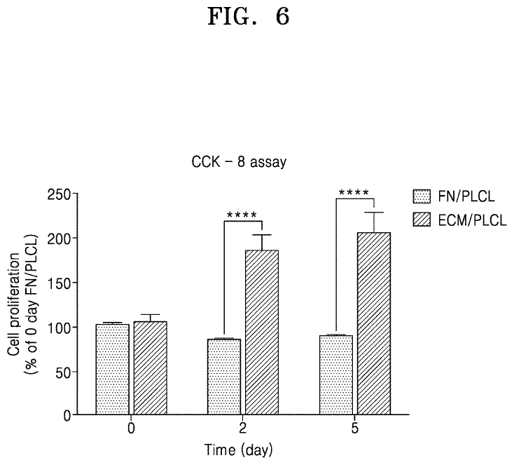

As a result, as shown in FIG. 6, the PLCL-ECM film was able to improve the proliferation ability of hCECs over time, and in particular, the cell proliferation level in the PLCL-ECM film was significantly improved, as compared with the control.

### Example 4. Comparison of Cell Proliferation Effects According to Polymer Materials

In this exemplary embodiment, cell proliferation effects were compared between the PLCL-ECM film according to one exemplary embodiment and a PVA-ECM film based on a polyvinyl alcohol (PVA) material. The PVA-ECM was produced in the same manner as in Example 1, and a CCK-8 assay was carried out using NIH3T3 cells in the same manner as in Example 3-3. Meanwhile, the measurement was expressed as a cell proliferation rate (%), based on a level of the cells present on the PVA-ECM film on day 0.

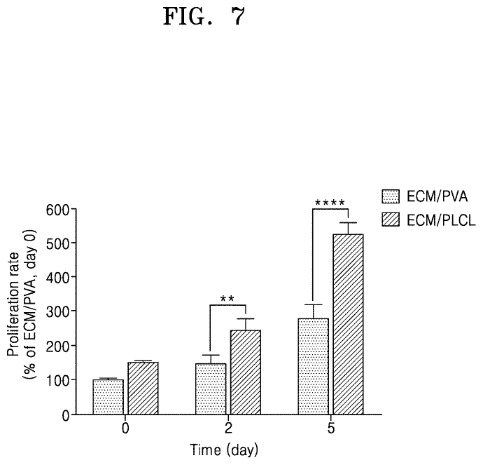

As a result, as shown in FIG. 7, the cell proliferation level in the PLCL-ECM film was significantly improved, as compared with the PVA-ECM film, and in particular, on day 5 after culture, the cell levels of the films showed a remarkable difference of more than twice.

A method according to an aspect may provide a polymer film having excellent biocompatibility and biological efficacy through a physical crosslinking reaction between poly(lactide-co-ε-caprolactone) and an extracellular matrix.

The poly(lactide-co-ε-caprolactone) film including an extracellular matrix according to an aspect may exhibit excellent cell adhesion ability and may also remarkably improve cell proliferation ability, thereby being applied to biological materials including an ophthalmic material in various fields.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A method of producing a poly(lactide-co-ε-caprolactone) film comprising an extracellular matrix, the method comprising:
producing a two-phase mixture comprising poly(lactide-co-ε-caprolactone) and an extracellular matrix by adding a poly(lactide-co-ε-caprolactone)(PLCL) polymer solution to a solid-phase substrate to which the extracellular matrix (ECM) is attached; and
forming a PLCL-ECM film on a surface of the solid-phase substrate by evaporating the solvent from the produced two-phase mixture,
wherein the PLCL-ECM film is formed by physical crosslinking between the PLCL and the ECM.

2. The method of claim 1, wherein the ECM is obtained through decellularization of a biological tissue or a cultured cell layer.

3. The method of claim 1, wherein the ECM is obtained through decellularization of a cultured fibroblast cell layer.

4. The method of claim 1, wherein a molecular weight of the PLCL is 100 kDa to 200 kDa.

5. The method of claim 1, wherein the PLCL solution comprises, as the solvent, chloroform, tetrahydrofuran, hexafluoroisopropanol, dimethylformamide, acetone, dimethyl sulfoxide, distilled water, a phosphate buffered solution (PBS), or saline.

6. The method of claim 1, wherein the PLCL solution has a concentration of 0.5% (w/v) to 5% (w/v).

7. The method of claim 1, wherein the evaporating of the solvent is carried out at 5° C. to 60° C.

8. The method of claim 7, wherein the evaporating of the solvent is carried out for 12 hr to 72 hr.

9. The method of claim 1, further comprising separating the PLCL-ECM film from the surface of the solid-phase substrate.

10. A poly(lactide-co-ε-caprolactone) film comprising an extracellular matrix, the film being produced by the method of claim 1.

11. The film of claim 10, wherein the film has a thickness of 5 μm to 25 μm.

12. The film of claim 10, wherein the film is used as an implant material.

13. A method of regenerating biological tissue, the method comprising administering or implanting, into an individual, a poly(lactide-co-ε-caprolactone) film comprising an extracellular matrix, the film being produced by the method of claim 1.

14. An ophthalmic material comprising the poly(lactide-co-ε-caprolactone) film comprising an extracellular matrix of claim 10.

15. The ophthalmic material of claim 14, wherein the ophthalmic material enhances survival and proliferation of corneal endothelial cells.
